(19) United States
(12) Patent Application Publication
(10) Pub. No.: US 2012/0165737 A1
(43) Pub. Date: Jun. 28, 2012

(54) DEVICES AND METHODS FOR MAINTAINING AN ASEPTIC CATHETER ENVIRONMENT

(75) Inventor: Nicholas B. Davis, Shelbyville, IN (US)

(73) Assignee: DAVIS & SON DEVELOPMENTS LLC, Shelbyville, IN (US)

(21) Appl. No.: 13/394,299

(22) PCT Filed: Sep. 2, 2010
(86) PCT No.: PCT/US10/47668

§ 371 Date: Mar. 5, 2012

Related U.S. Application Data
(60) Provisional application No. 61/239,629, filed on Sep. 3, 2009.

(51) Int. Cl. A61M 25/02; A61M 25/00

(52) U.S. Cl. 604/179; 604/523

(57) ABSTRACT

Devices and methods for maintaining an aseptic catheter environment. In at least one exemplary embodiment of a device for maintaining an aseptic catheter environment of the present disclosure, the device comprises a first housing and a second housing, the first housing and the second housing coupled to one another by way of at least one coupler, the first housing and the second housing configured to engage one another when in a closed configuration and to define an interior space when in the closed configuration, and at least one aperture and/or notch defined within at least one of the first housing and the second housing, the at least one aperture and/or notch sized and shaped to allow at least part of a catheter to be positioned therethrough.

### PRIORITY

The present application is related to, and claims the priority benefit of, U.S. Provisional Patent Application Ser. No. 61/239,629, filed Sep. 3, 2009, the contents of which are hereby incorporated by reference in their entirety into this disclosure.

### BACKGROUND

Infections stemming from unsanitary bodily catheters, including those used for dialysis, can both be costly and painful. Currently, when a patient or technician prepares his or her implanted bodily catheters for dialysis or another procedure, the external surfaces of the catheters must be cleaned prior to connection to dialysis or other machines. Improper or ineffective cleaning can lead to “dirty” connections, which often lead to costly, painful, life-threatening infections. Over five million central catheters are placed annually, resulting in 250,000 bloodstream infections. In the case of urinary catheters, such catheters result in 500,000 annual urinary tract infections.

Devices and methods useful to reduce or prevent bacterial or other infection in connection with various bodily catheters would solve this problem and be well received in the marketplace.

### BRIEF SUMMARY

The disclosure of the present application addresses the reduction and/or prevention of bacterial or other infection in connection with various bodily catheters, including those used for dialysis, such as central venous catheters as well as peripherally inserted central catheters (PICCs or PICC lines). Such bodily catheters may be used for various types of dialysis procedures, including, but not limited to, hemodialysis, perotineal dialysis, intestinal dialysis, hemodialysis, and other types of dialysis, such as liver dialysis, as well as for various injections and/or suction procedures, including those useful to remove excess fluid from various areas within the body. The devices of the present disclosure, for example, may be used by a patient to maintain an aseptic bodily catheter environment when not engaging in dialysis or when engaging in dialysis, as such devices “clean” the external portions of bodily catheters, dialyzer bloodline connectors, caps, etc., before, during, and after dialysis. Such devices may also provide the patient with more freedom to do typical tasks, such as showering, with a reduced risk of infection from said tasks. Such devices may also be relatively thin and lightweight to provide a comfortable experience when in use. In various embodiments, such devices may have rounded portions for additional comfort when the device comes in contact with a patient's body.

In an exemplary embodiment of a device for maintaining an aseptic catheter environment of the present disclosure, the device comprises a first housing and a second housing, the first and second housings coupled to one another by way of at least one coupler and configured to engage one another when in a closed configuration and to define an interior space when in the closed configuration, and at least one aperture and/or notch defined within the first housing and/or the second housing, said aperture sized and shaped to permit a catheter to be positioned therethrough.

In at least one exemplary embodiment of a device for maintaining an aseptic catheter environment of the present disclosure, the device comprises a first housing and a second housing, the first housing and the second housing coupled to one another by way of at least one coupler, the first housing and the second housing configured to engage one another when in a closed configuration and to define an interior space when in the closed configuration, and at least one aperture and/or notch defined within at least one of the first housing and the second housing, the at least one aperture and/or notch sized and shaped to allow at least part of a catheter to be positioned therethrough. In another embodiment, the at least one coupler comprises a hinge coupled to the first housing and the second housing so that the first housing and the second housing hingedly move about one another. In yet another embodiment, the at least one coupler comprises at least one clip, the at least one clip operable to reversibly secure the first housing to the second housing.

In at least one exemplary embodiment of a device for maintaining an aseptic catheter environment of the present disclosure, at least one of the first housing and the second housing comprises a housing gasket positioned around at least a portion of a perimeter of the first housing and/or the second housing. In an additional embodiment, the first housing comprises the housing gasket, and wherein the second housing comprises a groove positioned around at least a portion of a perimeter of the second housing, the groove configured to receive said housing gasket. In yet an additional embodiment, the housing gasket provides a fluid-tight seal when the first housing and the second housing engage each other in the closed configuration. In another embodiment, the device further comprises at least one aperture gasket positioned at or near the at least one aperture and/or notch, wherein the at least one aperture gasket provides a fluid-tight seal when a catheter is positioned within said aperture and/or notch.

In at least one exemplary embodiment of a device for maintaining an aseptic catheter environment of the present disclosure, the device further comprises one or more saturable materials positioned within the interior space of the device. In an additional embodiment, the one or more saturable materials are selected from the group consisting of a sponge, a fabric, and/or a gauze.

In at least one exemplary embodiment of a device for maintaining an aseptic catheter environment of the present disclosure, the device further comprises a fluid sac containing an antiseptic fluid, the fluid sac positioned upon at least one of the first housing and the second housing. In another embodiment, the fluid sac is capable of being ruptured by way of a rupturer positioned within said device. In various embodiments, the rupturer is selected from the group consisting of a pin, a needle, a portion of the first housing, and a portion of the second housing. In an additional embodiment, and when the fluid sac becomes ruptured, the fluid sac releases fluid within the interior space of the device to create an aseptic environment within the interior space of the device. In yet an additional embodiment, the fluid sac is capable of being ruptured by way of closing the device, whereby pressure from at least one of the first housing and the second housing causes the fluid sac to rupture. In another embodiment, and when the fluid sac becomes ruptured, the fluid sac releases fluid within the interior space of the device to create an aseptic environment within the interior space of the device.

In at least one exemplary embodiment of a device for maintaining an aseptic catheter environment of the present disclosure, the at least one aperture and/or notch comprises at least two apertures and/or notches. In an additional embodiment, the at least one aperture and/or notch comprises at least four apertures and/or notches, wherein at least two of the at least four apertures and/or notches are positioned relatively opposite to at least two other of the at least four apertures and/or notches. In yet an additional embodiment, the device further comprises a strap coupled thereto, the strap configured to be positioned about a portion of a patient's body. In another embodiment, the strap further comprises a strap adjuster to allow the patient to adjust the strap.

In at least one exemplary embodiment of a device for maintaining an aseptic catheter environment of the present disclosure, the device comprises a first housing and a second housing, the first housing and the second housing coupled to one another by way of at least one hinge, the first housing and the second housing configured to engage one another when in a closed configuration and to define an interior space when in the closed configuration, at least one clip coupled to the first housing or the second housing, the at least one clip operable to reversibly secure the first housing to the second housing, one or more saturable materials positioned within the interior space of the device, a fluid sac containing an antiseptic fluid, the fluid sac positioned upon at least one of the first housing and the second housing, and at least one aperture and/or notch defined within at least one of the first housing and the second housing, the at least one aperture and/or notch sized and shaped to allow at least a portion of a catheter to be positioned therethrough.

In at least one exemplary embodiment of a device for maintaining an aseptic catheter environment of the present disclosure, the device comprises a first housing and a second housing, the first housing and the second housing coupled to one another by way of at least one hinge, the first housing and the second housing configured to engage one another when in a closed configuration and to define an interior space when in the closed configuration, at least one clip coupled to the first housing or the second housing, the at least one clip operable to reversibly secure the first housing to the second housing, one or more saturable materials positioned within the interior space of the device, a fluid sac containing an antiseptic fluid, the fluid sac positioned upon at least one of the first housing and the second housing, and at least four apertures and/or notches defined within at least one of the first housing and the second housing, wherein at least two of the at least four apertures and/or notches are positioned relatively opposite to at least two other of the at least four apertures and/or notches, the at least four apertures and/or notches sized and shaped to allow at least portions of catheters to be positioned therethrough.

In at least one exemplary embodiment of a method of using a device for maintaining an aseptic catheter environment of the present disclosure, the method comprises the steps of placing a distal end of a bodily catheter within a aperture and/or a notch of a device for maintaining an aseptic catheter environment, closing said device, and activating said device to release an antiseptic fluid within said device. In another embodiment, the step of activating said device is performed by applying pressure to the device to cause the antiseptic fluid within said device to be released.

In at least one exemplary embodiment of a method of proceeding with dialysis using a device for maintaining an aseptic catheter environment of the present disclosure, the method comprises the steps of removing a device for maintaining an aseptic catheter environment from one or more bodily catheters positioned at least partially therein, removing caps positioned at or near a distal end of the one or more bodily catheters, connecting the one or more bodily catheters to one or more dialyzer catheters, removing clamps positioned upon the one or more bodily catheters, and proceeding with dialysis.

In at least one exemplary embodiment of a method of proceeding with dialysis using a device for maintaining an aseptic catheter environment of the present disclosure, the method comprises the steps of removing caps positioned at or near the distal ends of bodily catheters, connecting the bodily catheters to dialyzer catheters, positioning a device for maintaining an aseptic catheter environment about at least a portion of the bodily catheters and at least a portion of the dialyzer catheters, closing said device, activating said device to release an antiseptic fluid within said device, removing clamps positioned upon the bodily catheters, and proceeding with dialysis.

In at least one exemplary embodiment of an adhesive device of the present disclosure, the device comprises a pouch sized and shaped to permit a portion of a bodily catheter extending from a patient's body to be positioned therein, and an adhesive ring coupled to said pouch, wherein when the bodily catheter is positioned within the pouch, the adhesive ring is capable of adhering to the patient's body to form at least a substantially fluid and/or air tight seal.

### BRIEF DESCRIPTION OF THE DRAWINGS

FIG. 1A shows a front view of an exemplary embodiment of a device for maintaining an aseptic catheter environment according to the present disclosure;

FIGS. 1B and 1C show side views of an exemplary device according to an embodiment of the present disclosure;

FIG. 1D shows a top view of an exemplary device according to an embodiment of the present disclosure;

FIGS. 2A and 2B show views of open devices for maintaining an aseptic catheter environment according to embodiments of the present disclosure;

FIGS. 2C and 2D show cross-sectional views of a device for maintaining an aseptic catheter environment according to embodiments of the present disclosure;

FIG. 2E shows steps of an exemplary method for using an exemplary device for maintaining an aseptic catheter environment according to an embodiment of the present disclosure;

FIG. 2F shows a view of an open device for maintaining an aseptic catheter environment with bodily catheters positioned therein according to an embodiment of the present disclosure;

FIG. 2G shows a view of a closed device for maintaining an aseptic catheter environment with bodily catheters positioned therein according to an embodiment of the present disclosure;

FIG. 2H shows steps of an exemplary method for proceeding with dialysis after using an exemplary device for maintaining an aseptic catheter environment according to an embodiment of the present disclosure;

FIG. 3A shows a front view of an exemplary embodiment of device for maintaining an aseptic catheter environment according to an embodiment of the present disclosure;

FIGS. 3B and 3C show side views of an exemplary device according to an embodiment of the present disclosure;

FIG. 3D shows a top view of an exemplary device according to an embodiment of the present disclosure;

FIG. 3E shows a bottom view of an exemplary device according to an embodiment of the present disclosure;

FIG. 4 shows a view of an open device for maintaining an aseptic catheter environment with bodily catheters and dialyzer catheters positioned therein according to an embodiment of the present disclosure;

FIG. 5 shows steps of an exemplary method for using an exemplary device for maintaining an aseptic catheter environment according to an embodiment of the present disclosure;

FIG. 6 shows a front view of an exemplary embodiment of a device for maintaining an aseptic catheter environment with a strap coupled thereto according to an embodiment of the present disclosure;

FIG. 7 shows an exemplary adhesive device according to an embodiment of the present disclosure; and

FIG. 8 shows an exemplary adhesive device with a bodily catheter positioned therein according to an embodiment of the present disclosure.

### DETAILED DESCRIPTION

The disclosure of the present application provides various devices and methods for maintaining an aseptic catheter environment. For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

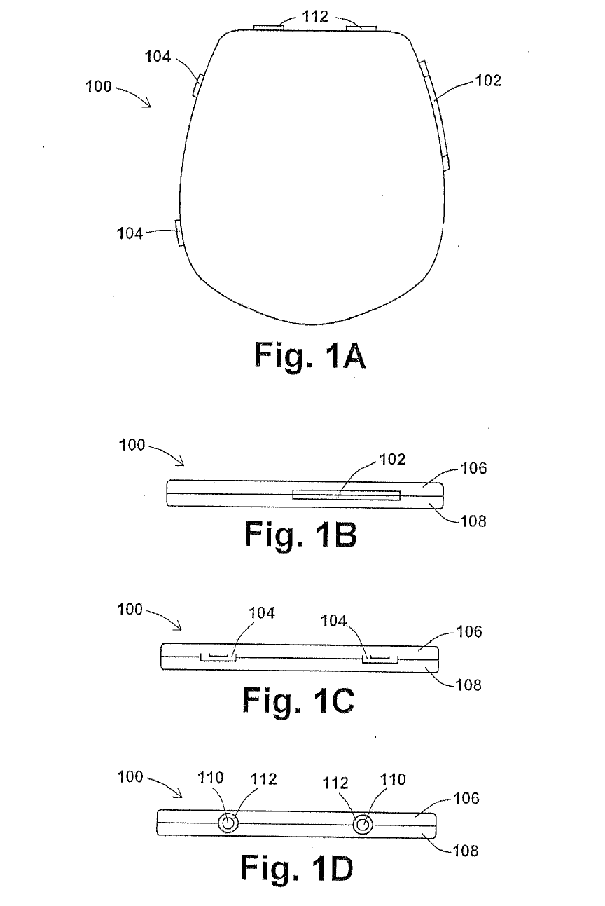

An exemplary embodiment of a device for maintaining an aseptic catheter environment of the present disclosure is shown in FIGS. 1A-1D. As shown in the front view shown in FIG. 1A, an exemplary embodiment of device 100 comprises a hinge 102 and one or more clips 104. Hinge 102 and clips 104, in at least one embodiment, are coupled to device 100 opposite one another to facilitate the opening, closing, and securing of a first housing 106 and a second housing 108 to one another as shown in FIGS. 1B-1D. As shown in FIGS. 1A-1D, device 100 is in a “closed” configuration, whereby first housing 106 and second housing 108 define a volume within device 100.

First housing 106, second housing 108, hinge 102, and clips 104 may comprise any number of materials known in the art suitable for an exemplary device 100 as referenced herein including, but not limited to, various plastics and/or metals. In at least one embodiment, first housing 106, second housing 108, hinge 102, and clips 104 each comprise polypropylene plastic. Said materials, in at least one embodiment, should be of sufficient rigidity so that the various embodiments of devices 100 of the present disclosure may sufficiently operate, for example, to remain closed when bodily catheters are positioned within device 100, and to form an appropriate seal when device 100 is in a “closed” configuration. In at least one embodiment, first housing 106, second housing 108, and hinge 102 are unitary in nature by way of being formed as one molded plastic piece, for example.

FIG. 1B shows a side view of an exemplary device 100 of the present disclosure. As shown in FIG. 1B, device 100 comprises a first housing 106 and a second housing 108, whereby first housing 106 and second housing 108 are coupled to one another by way of hinge 102. Hinge 102 may comprise any number of hinge mechanisms known in the art, including, but not limited to, a pliable plastic hinge, so that hinge 102 permits first housing 106 and second housing 108 to hingedly move in relation to one another.

FIG. 1C shows a side view of an exemplary device 100 of the present disclosure. As shown in FIG. 1C, device 100 comprises a first housing 106 and a second housing 108, whereby first housing 106 and second housing 108 may be reversibly secured to one another by way of one or more clips 104. Clips 104, as shown in FIG. 1C, may comprise clips extending from either the first housing 106 and the second housing 108, and operate to reversibly secure the first housing 106 to the second housing 108. For purposes of the present disclosure, hinge 102 and clips 104 may be generally referred to as a “coupler” or “couplers.” In such an embodiment, for example, clips 104, and not a hinge 102, is used to couple first housing 106 to second housing 108.

A top view of an exemplary embodiment of a device 100 of the present disclosure is shown in FIG. 1D. As shown in FIG. 1D, device 100 defines at least one aperture 110 positioned therethrough. In the exemplary embodiment shown in FIG. 1D, apertures 110 are partly defined by first housing 106 and partly defined by second housing 108. In another embodiment, apertures 110 may be fully defined within either first housing 106 and/or second housing 108. In addition, and as shown in FIGS. 1A and 1D, device 100 may further comprise one or more aperture gaskets 112 positioned at or near apertures 110, whereby aperture gaskets 112 facilitate a fluid-tight seal at the site of aperture(s) 110 when, for example, a catheter (not shown) is positioned therethrough as discussed hereinafter.

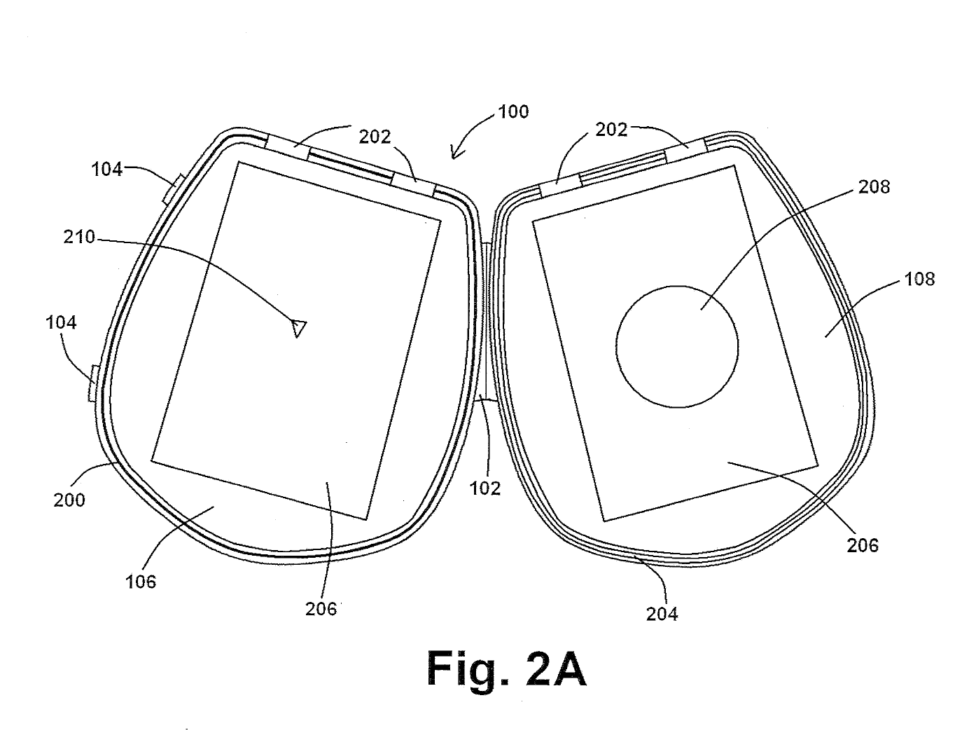

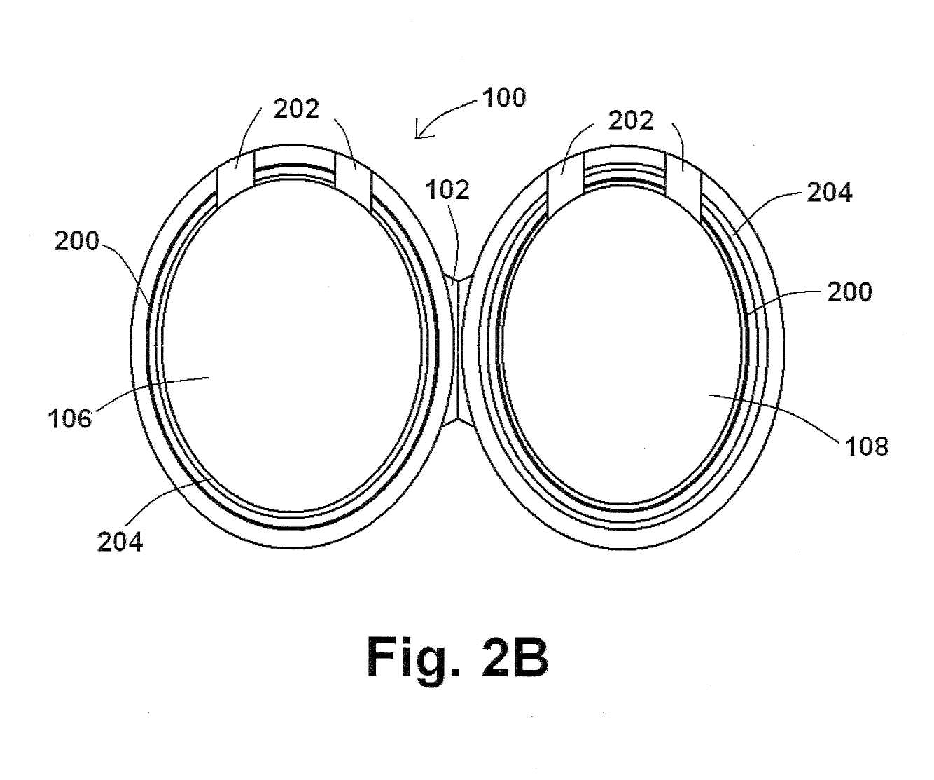

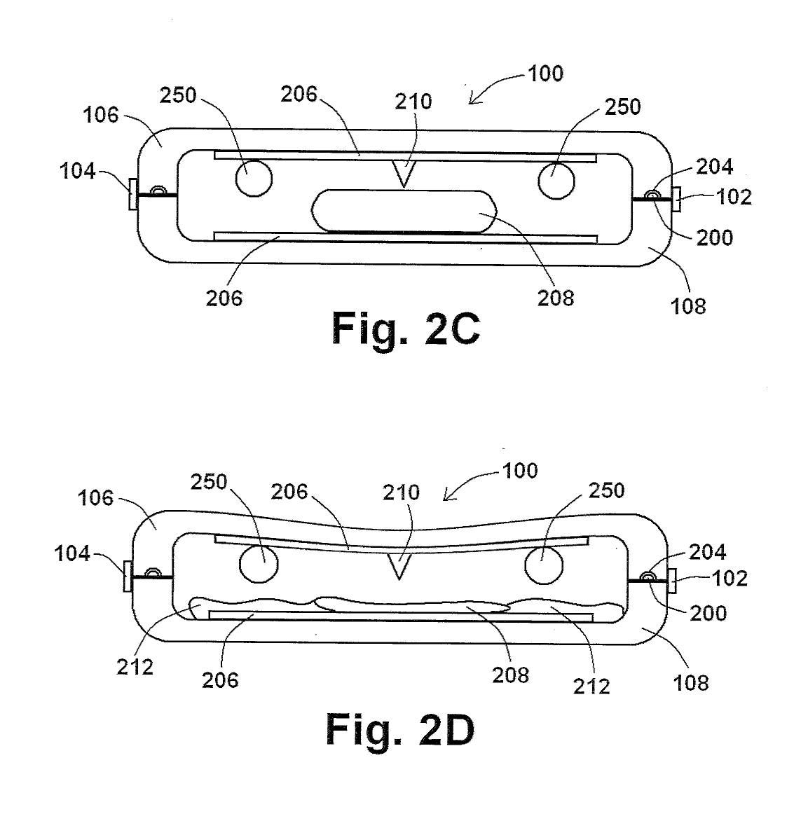

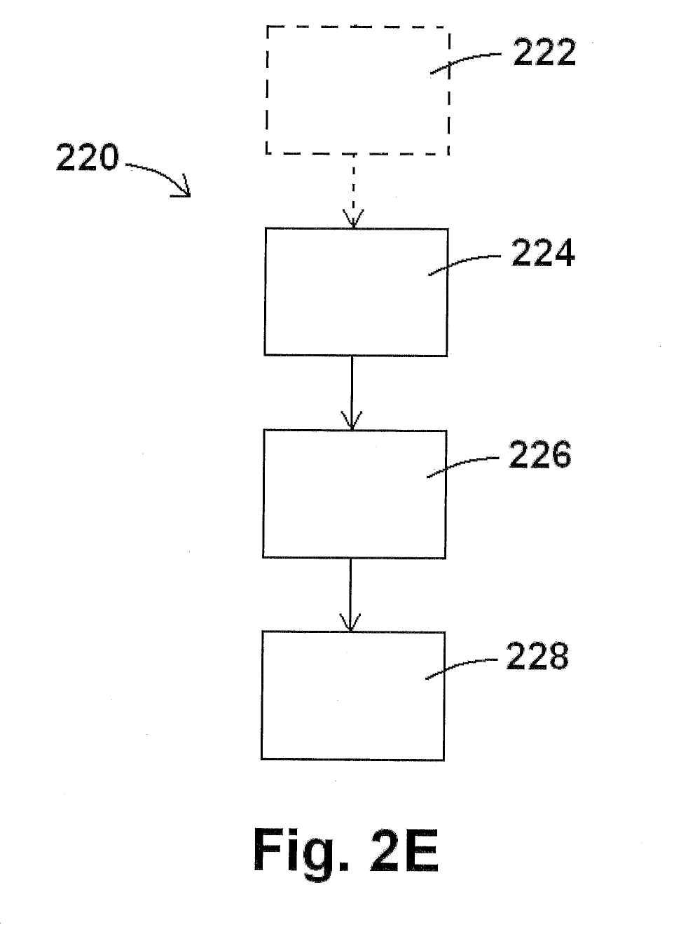

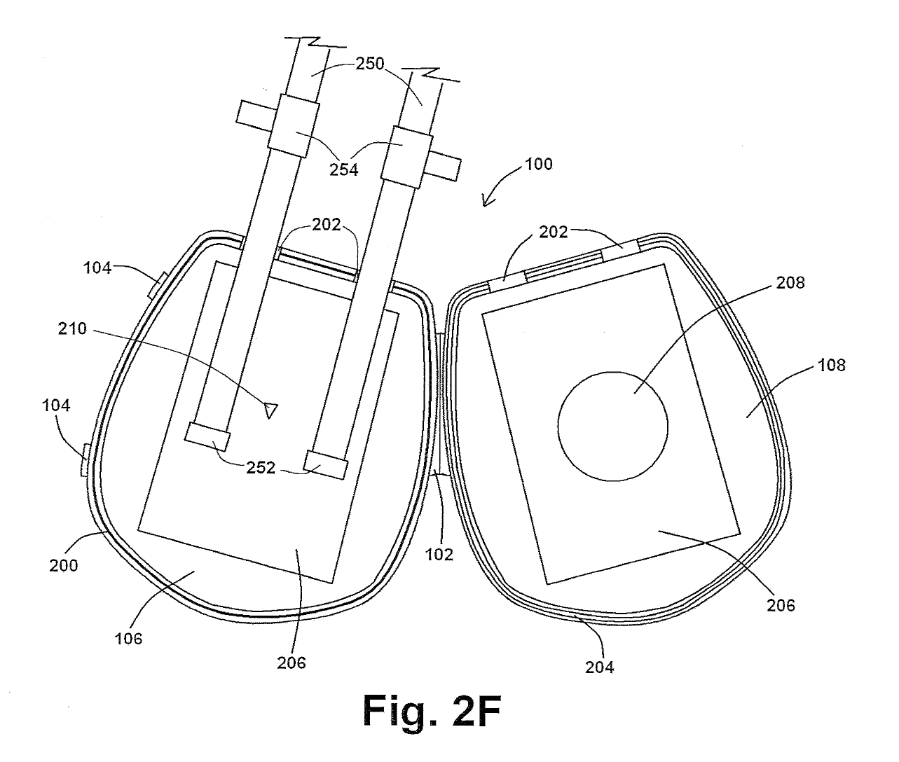

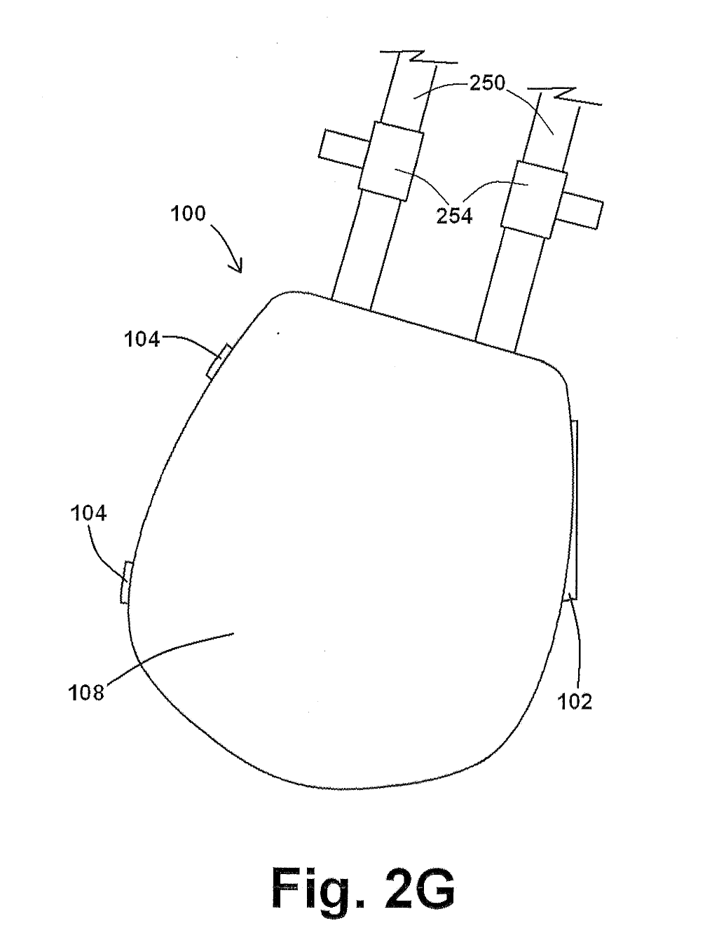

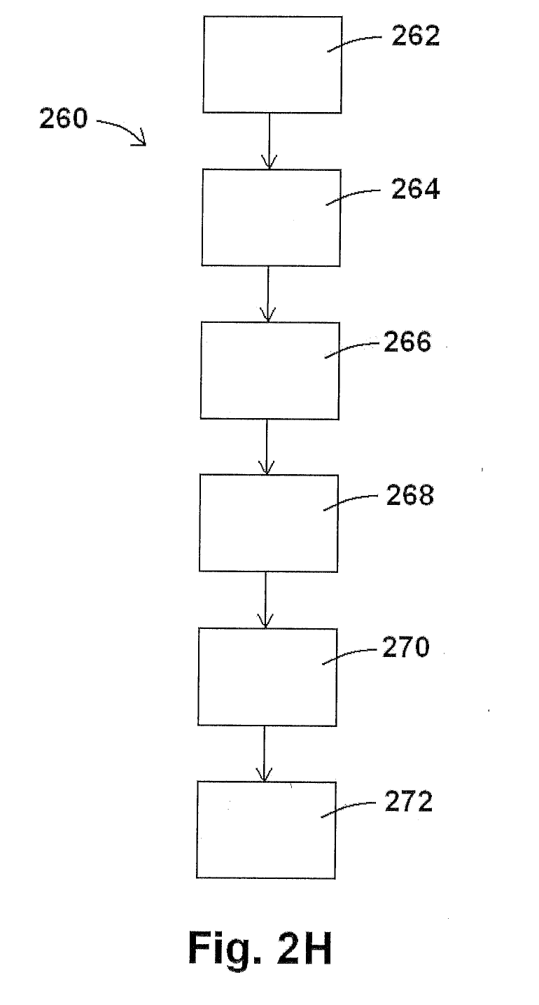

An open view of an exemplary embodiment of a device 100 of the present disclosure is shown in FIG. 2A. As shown in FIG. 2A, device 100 may comprise a first housing 106 coupled to a second housing 108 by way of hinge 102, whereby clips 104 positioned along first housing 106 may operate to engage second housing 108 when device 100 is in a “closed” configuration. Alternative embodiments, as discussed herein, may appear similar to the exemplary embodiment of device 100 shown in FIG. 2A but may include, for example, additional clips 104 in lieu of hinge 102, so that clips 104 may reversibly secure first housing 106 to second housing 108. Furthermore, clips 104 may appear on either or both first housing 106 and/or second housing 108.

As shown in FIG. 2A, device 100 may further comprise an optional housing gasket 200 positioned around at least part of the perimeter of first housing 106 and/or second housing 108. Housing gasket 200, when device 100 is in a “closed” configuration, facilitates a fluid-tight or substantially fluid-tight seal so that fluid positioned within device 100 will not leak outside device 100. Such a fluid tight seal may exist in connection with a fluid tight seal present by way of catheters being positioned within apertures 110, shown in FIG. 2A as being defined by notches 202 positioned within first housing 106 and second housing 108, so that when device 100 is in a “closed” configuration, apertures 110 are defined by way of notches 202. Aperture gaskets 112 (shown in FIG. 1D), and in an exemplary embodiment, may be positioned at or near notches 202 (as shown in FIG. 2A) to further facilitate a fluid-tight seal when, for example, a catheter is positioned therethrough and in contact therewith.

In addition to the foregoing, one or more grooves 204 may be positioned in a housing opposite the housing comprising housing gasket 200, whereby housing gasket 200 may engage groove 204 when the device is in a “closed” configuration. In at least one embodiment, and as shown in FIG. 2B, each housing comprises a housing gasket 200 and a groove 204, so that the housing gasket 200 of first housing 106 engages groove 204 of second housing 108, and housing gasket 200 of second housing 108 engages groove 204 of first housing 106, forming an fluid and/or air tight seal.

In the embodiment shown in FIG. 2A, device 100 further comprises one or more saturable materials 206 positioned “inside” of device 100. FIG. 2A shows saturable materials 206 positioned upon first housing 106 and second housing 108, but any number of saturable materials 206 may be used, including one saturable material 206. Saturable materials 206 may comprise any number of materials that can become either partially or fully saturated by a fluid, such as, for example, a sponge, a fabric, or a gauze, and may either be initially provided “dry” (i.e., without any fluid) or “wet” (i.e., with, saline, an antiseptic fluid, etc., positioned thereon).

Such saturable materials 206 may become either partially or fully saturated by a fluid initially positioned within, for example, fluid sac 208 as shown in FIG. 2A. Fluid sac 208, in at least one embodiment, may be ruptured by way of one or more rupturers 210, which may comprise, for example, a pin or a needle coupled to first housing 106 and/or second housing 108. In alternative embodiments, rupturer 210 may comprise a relatively “sharp” portion protruding from first housing 106 and/or second housing 108, or may comprise a portion of first housing 106 and/or second housing 108 which is not relatively “sharp” but is otherwise flexible/pliable so that when a user of device 100 exerts pressure on rupturer 210, rupturer 210 facilitates the rupture of fluid sac 208. As shown in the exemplary embodiment of device 100 shown in FIG. 2A, rupturer 210 is coupled to, or formed as a part of, first housing 106. In at least one embodiment, rupturer 210 is positioned within device 100 at a location so not to potentially puncture any bodily catheter positioned therein. Such puncture prevention may be accomplished in several ways including, but not limited to, device 100 design elements, grooves within saturable elements 206 to receive the catheters, and/or physical barriers within device 100 to prevent movement of the catheters within device 100.

In at least one embodiment of a device 100 of the present disclosure, the closure of said device 100 itself causes fluid sac 208 to rupture. For example, and in at least one embodiment, fluid sac 208 is sized and shaped and contains enough fluid so that when device 100 is closed (with our without portions of catheters therein), the closure causes pressure to be exerted upon fluid sac 208 causing fluid sac to rupture.

Fluid within fluid sac 208 may comprise, for example, any number of antiseptic fluids including, but not limited to, betadine, iodine, various alcohols, various chlorines and/or hypochlorites, chlorhexidine, hypoallergenic solutions, and/or any other antibacterial agents known in the art. Such fluids, when released from fluid sac 208, can enter an interior space of device 100 (created when device 100 is in a “closed” configuration), and can serve as an antiseptic/antibacterial agent and effectively create an “aseptic” environment within device 100.

FIGS. 2C and 2D show a cross-sectional view of an exemplary device 100 of the present disclosure. As shown in FIG. 2C, device 100 comprises a rupturer 210 coupled/affixed to first housing 106, positioned relative to a fluid sac 208 present within the space between first housing 106 and second housing 108. When device 100 is “closed,” as shown in FIG. 2C, housing gasket 200 is positioned within groove 204, forming a fluid and/or air tight seal (or substantially a fluid and/or air tight seal). Squeezing first housing 106, for example and as shown by way of the deflection of first housing 106 shown in FIG. 2D, causes rupturer 210 to rupture fluid sac 208, thereby releasing fluid 212 from fluid sac 208 into the volume/space within the closed device 100. Fluid 212 may then be absorbed by saturable material(s) 206 present within device 100 and also coat some or all of bodily catheters 250 positioned within device 100, creating an aseptic environment as referenced herein. Such an embodiment of device 100 shown in FIGS. 2C and 2D may be referred to as a “single-use device,” as fluid sac 208 is ruptured to release fluid 212 within device 100.

An exemplary method of using a device 100 of the present application is shown in FIG. 2E and described below. For example, a device 100 as shown in FIGS. 1A-2A may be used by a dialysis patient having one or more catheters extending from the patient's body which are in communication with the patients circulatory system (e.g., a patient's jugular vein, subclavian vein, vena cava, etc.). Such catheters may have single or multiple lumens. In the exemplary embodiment of device 100 shown in FIGS. 1A-2A, the patient has one bodily catheter (with a double lumen) that branches into two catheters (one for blood/fluid flow from the patient's body and one for blood/fluid flow back into the patient's body to the dialyzer after the blood/fluid is processed through a dialyzer), or the patient may have two bodily catheters extending from his or her body serving a similar purpose as described herein.

In an exemplary method 220 of the present disclosure, and as shown in FIG. 2E, prior to a new dialysis session, or sometime after a prior dialysis session, a patient may optionally “clean” his or her bodily catheters by any number of means known in the art (optional cleaning step 222) to remove and/or neutralize, for example, excess bacteria around the bodily catheters. Regardless of prior catheter cleaning, the patient, or a user of device 100, may then place device 100 about his or her bodily catheters by placing the distal ends of the catheters within apertures 110 or notches 202 of device 100 so that the exposed ends of said catheters are positioned within device 100 (catheter placement step 224). After the bodily catheters are positioned within device 100, device 100 may be “closed” about the distal ends of the bodily catheters (closing step 226). After device 100 is “closed,” the patient, and/or a user of device 100, may “activate” device 100 by, for example, squeezing device 100 so that rupturer 210 may rupture fluid sac 208 to release antiseptic fluid within device 100 (activation step 228), creating an aseptic environment. After the antiseptic fluid present within device 100 has been in contact with the distal ends of the bodily catheters, the patient shall be ready to proceed with dialysis without the need to perform any additional/extensive bodily catheter cleaning procedures as the bodily catheters will already be “clean,” thus reducing the likelihood/risk of bacterial or other infection common when connecting the bodily dialysis catheters to a dialyzer. Removal of device 100, in at least one embodiment, shall comprise the steps of “opening” device 100 and removing device 100 from the bodily catheters, thereby allowing the patient to connect the bodily catheters to a dialyzer to proceed with dialysis.

An embodiment of device 100 of the present disclosure showing bodily catheters 250 positioned therein is shown in FIG. 2F. As shown in FIG. 2F, the distal ends of bodily catheters 250 may be positioned within notches 202 (or apertures 110) of device 100, with the bodily catheters 250 being capped using, for example, caps 252, and optionally clamped using, for example, clamps 254. Caps 252 and/or clamps 254 may function to prevent the flow of fluid (blood in the case of hemodialysis) from a patient's body out of bodily catheters 250. Upon “closing” and “activating” device 100 as described above, the distal ends of bodily catheters 250, including caps 252, shall be within an aseptic environment created by device 100, thus reducing the risk of potential infection when the patient proceeds with a dialysis procedure as described below. Device 100, shown in an “open” configuration in FIG. 2F, may be “closed” as shown in FIG. 2G, whereby bodily catheters 250 are positioned within the interior space/volume of device 100.

In an exemplary method 260 of proceeding with dialysis of the present disclosure, and as shown in FIG. 2G, a patient, or a user of device 100, may remove device 100 from bodily catheters 250 (removal step 262) and prepare a dialyzer for dialysis (dialyzer preparation step 264, which may include any number of tasks known in connection with performing dialysis) prior to proceeding with dialysis. Removal step 262 and dialyzer preparation step 264 may be performed in either order, but are shown in the exemplary method shown in FIG. 2G as having removal step 262 precede dialyzer preparation step 264. The patient, or a user of device 100, may then remove caps 252 (leaving clamps 254 in place to prevent fluid flow from the patient's body) (cap removal step 266), and then connect bodily catheters 250 to the corresponding dialzyer catheters/connectors (connection step 268). The patient, or a user of device 100, may then remove clamps 254 (clamp removal step 270) and proceed with dialysis (dialysis step 272).

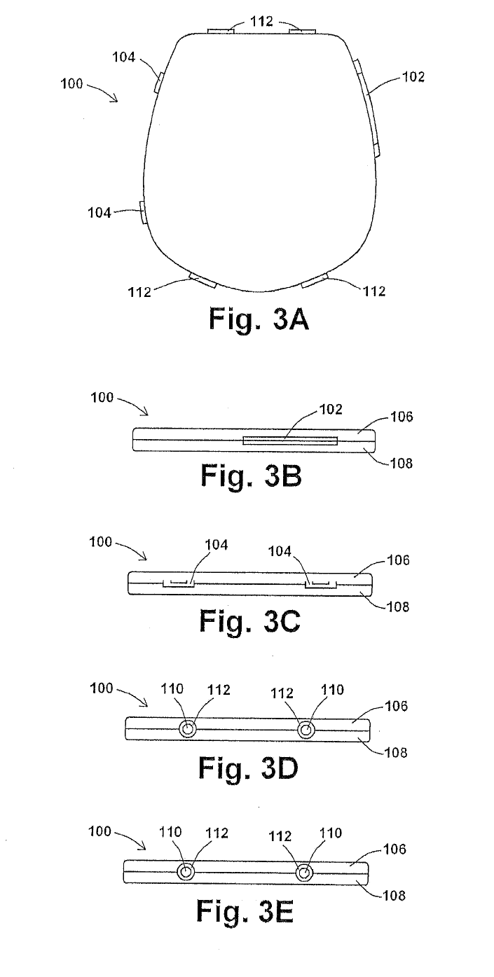

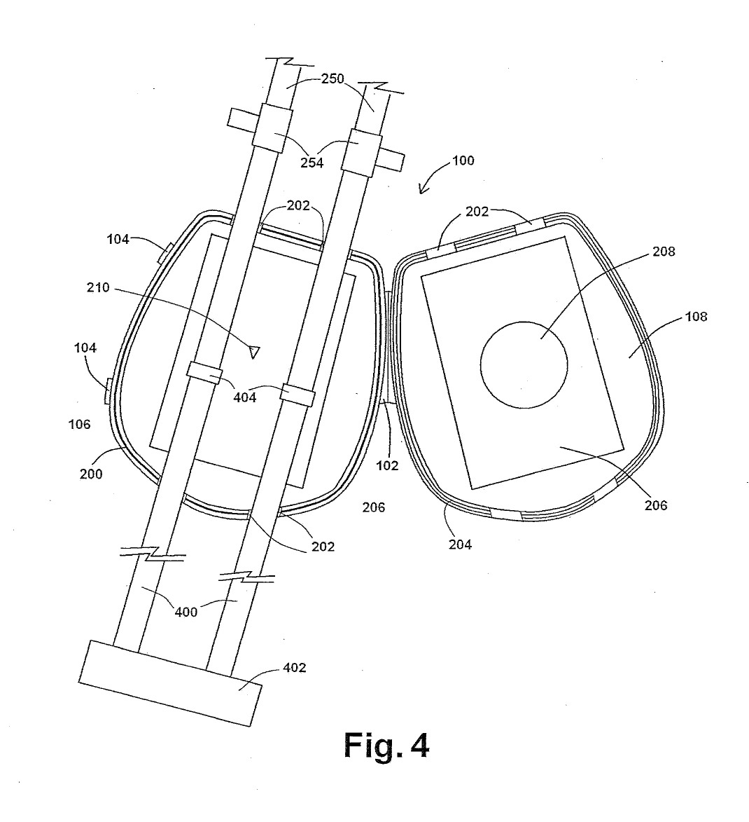

Another exemplary embodiment of a device for maintaining an aseptic catheter environment of the present disclosure is shown in FIGS. 3A-3E. As shown in the front view shown in FIG. 3A, an exemplary embodiment of device 100 may comprise a hinge 102 and one or more clips 104. Hinge 102 and clips 104, in at least one embodiment, are coupled to device 100 opposite one another to facilitate the opening, closing, and securing of a first housing 106 and a second housing 108 to one another as shown in FIGS. 3B-3E and 4. First housing 106, second housing 108, hinge 102, and clips 104 may comprise any number of materials known in the art suitable for an exemplary device 100 as referenced herein including, but not limited to, various plastics and/or metals. In at least one embodiment, first housing 106, second housing 108, hinge 102, and clips 104 each comprise polypropylene plastic.

FIG. 3B shows a side view of an exemplary device 100 of the present disclosure. As shown in FIG. 3B, device 100 may comprise a first housing 106 and a second housing 108, whereby first housing 106 and second housing 108 are coupled to one another by way of hinge 102. Hinge 102 may comprise any number of hinge mechanisms known in the art, including, but not limited to, a pliable plastic hinge, so that hinge 102 permits first housing 106 and second housing 108 to hingedly move about one another.

FIG. 3C shows a side view of an exemplary device 100 of the present disclosure. As shown in FIG. 3C, device 100 may comprise a first housing 106 and a second housing 108, whereby first housing 106 and second housing 108 may be reversibly secured to one another by way of one or more clips 104. Clips 104, as shown in FIG. 3C, may comprise clips extending from either the first housing 106 and the second housing 108, and operate to reversibly secure the first housing 106 to the second housing 108. For purposes of the present disclosure, hinge 102 and clips 104 may be generally referred to as a “coupler” or “couplers.”

A top view of an exemplary embodiment of a device 100 of the present disclosure is shown in FIG. 3D, and a bottom view of an exemplary embodiment of a device 100 of the present disclosure is shown in FIG. 3E. As shown in FIGS. 3D and 3E, device 100 defines four apertures 110 positioned therethrough (two apertures 110 at the top and two apertures at the bottom). In an embodiment whereby the bodily catheters 250 or the dialyzer catheters comprise a multi lumen catheter, an exemplary embodiment of device 100 may comprise one aperture positioned at the top of device 100 and one aperture positioned at the bottom of device 100. The number of apertures 110, and/or the physical placement of apertures about device 100, may vary from embodiment to embodiment. In the exemplary embodiments shown in FIGS. 3D and 3E, apertures 110 are partly defined by first housing 106 and partly defined by second housing 108. In other embodiments, apertures 110 may be fully defined within either first housing 106 and/or second housing 108. In addition, and as shown in FIGS. 3A, 3D, and 3E, device 100 may further comprise one or more aperture gaskets 112 positioned at or near apertures 110, whereby aperture gaskets 112 facilitate the presence of a “fluid-tight” seal at the site of aperture(s) 110 when, for example, a catheter (not shown) is positioned therethrough.

An embodiment of device 100 of the present disclosure showing bodily catheters 250 and dialyzer catheters 400 positioned therein is shown in FIG. 4. As shown in FIG. 4, the distal ends of bodily catheters 250 may be positioned within notches 202 (or apertures 110) of device 100, with the bodily catheters 250 being clamped using, for example, clamps 254. Furthermore, the distal ends of dialyzer catheters 400 of dialyzer 402 may be connected to the distal ends of bodily catheters 250 using, for example, connector 404 as shown in FIG. 4, so that the internal lumens of the bodily catheters 250 and the dialyzer catheters 400 are in fluid communication with one another. Upon “closing” and “activating” device 100 as described above, the distal ends of bodily catheters 250 and dialyzer catheters 400, including connector 404 if used to connect bodily catheters 250 to dialyzer catheters 400, shall be within an aseptic environment created by device 100, thus reducing the risk of potential infection when the patient proceeds with a dialysis procedure as described below.

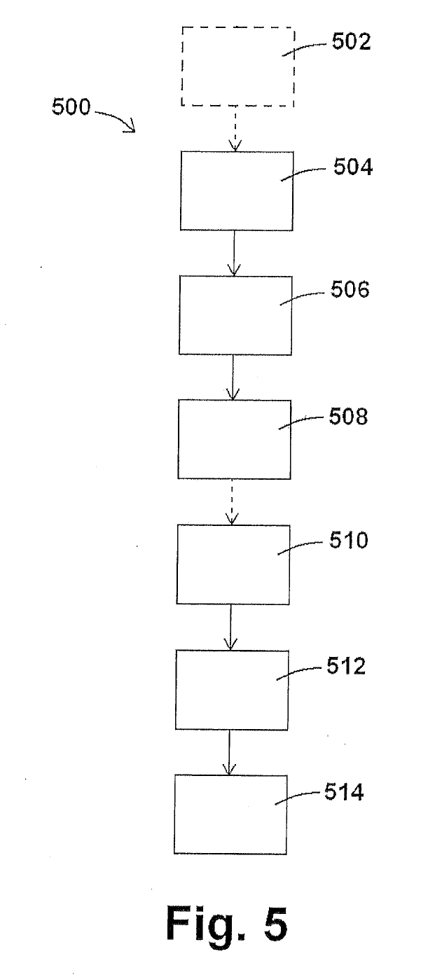

In an exemplary method 500 of proceeding with dialysis of the present disclosure, and as shown in FIG. 5, a patient or technician may optionally “clean” his or her bodily catheters by any number of means known in the art (optional cleaning step 502) to remove and/or neutralize, for example, excess bacteria around the bodily catheters. Regardless of prior catheter cleaning, the patient or technician may remove caps 252 from bodily catheters 250 (leaving clamps 254 in place to prevent fluid flow from the patient's body) (cap removal step 504), and then connect bodily catheters 250 to the corresponding dialzyer catheters 400 (connection step 506). After the bodily catheters 250 are connected to the dialyzer catheters 400 and are positioned within device 100, device 100 may be “closed” about the distal ends of the bodily catheters (closing step 508). After device 100 is “closed,” the patient may “activate” device 100 by, for example, pressing device 100 so that rupturer 210 may rupture fluid sac 208 to release antiseptic fluid within device 100 (activation step 510), creating an aseptic environment. After a desired amount of time has lapsed (so that the antiseptic fluid present within device 100 has been in contact with the distal ends of the bodily catheters 250 and the distal ends of dialyzer catheters 400), the patient shall be ready to proceed with dialysis without the need to perform any additional/extensive bodily catheter 250 and/or dialyzer catheter 400 cleaning procedures those catheters will already be “clean,” thus reducing the likelihood/risk of bacterial or other infection common when performing dialysis. The patient or technician may then remove clamps 254 (clamp removal step 512) and proceed with dialysis (dialysis step 514). Upon completion of dialysis, the patient or technician may remove device 100 (using the reverse of one or more method 500 steps herein), and optionally affix an embodiment of device 100 to his or her bodily catheters (as shown in FIG. 1A) to maintain an aseptic environment when not engaging in dialysis and/or any other medical procedure.

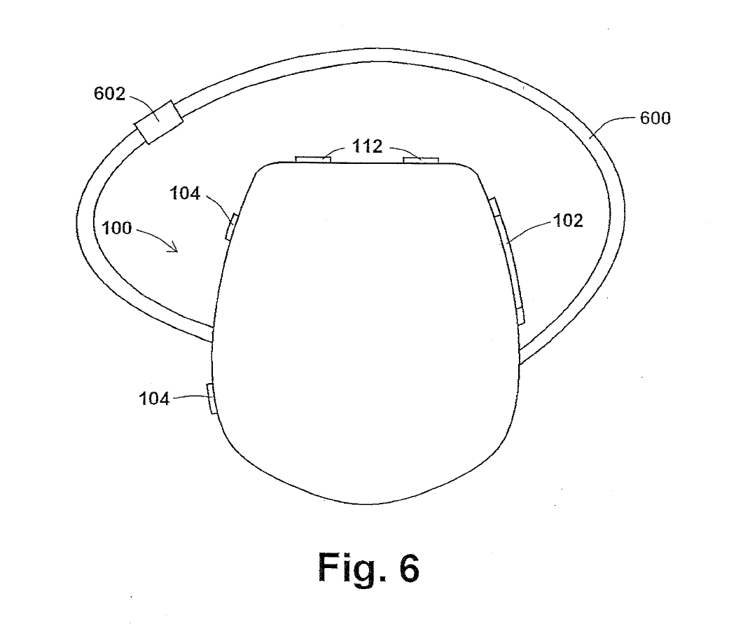

An exemplary embodiment of a device 100 of the present disclosure may be used with one or more straps 600 coupled thereto as shown in FIG. 6. As shown in FIG. 6, device 100 comprises strap 600 coupled thereto, whereby strap 600 is configured to be positioned around a portion of a patient's body, such as the patient's torso. Strap 600 may comprise any number of straps known in the art, including elastic straps, and may be padded for comfort. An exemplary embodiment of a strap 600 may also comprise a strap adjuster 602 to allow the patient to adjust strap 600 as desired. In lieu of the use of strap 600, or in connection with said use, devices 100 may also be removably adhered to a patient's body using one or more adhesives known in the art. Various exemplary embodiments of devices 100 of the present disclosure may incorporate a strap 600 as desired by a user.

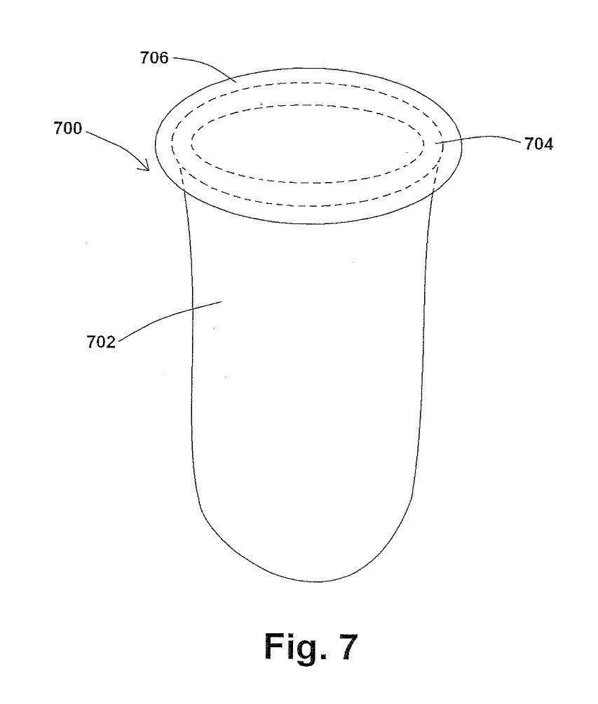

An exemplary embodiment of an adhesive device 700 of the present disclosure is shown in FIG. 7. As shown in FIG. 7, adhesive device comprises a pouch 702 and an adhesive ring 704 coupled to pouch 702. A patient may insert the distal ends of one or more bodily catheters 250 into said pouch 702, and by applying pressure to adhesive ring 704 about a patient's body, adhesive ring 704 may adhere the patient's body to provide an environment within pouch 702 that either reduces or prevents the introduction of fluids, particles, and/or air from entering pouch 702. Adhesive ring 704 may be covered by removable cover 706, such that removal of removable cover 706 exposes an adhesive positioned about adhesive ring 704.

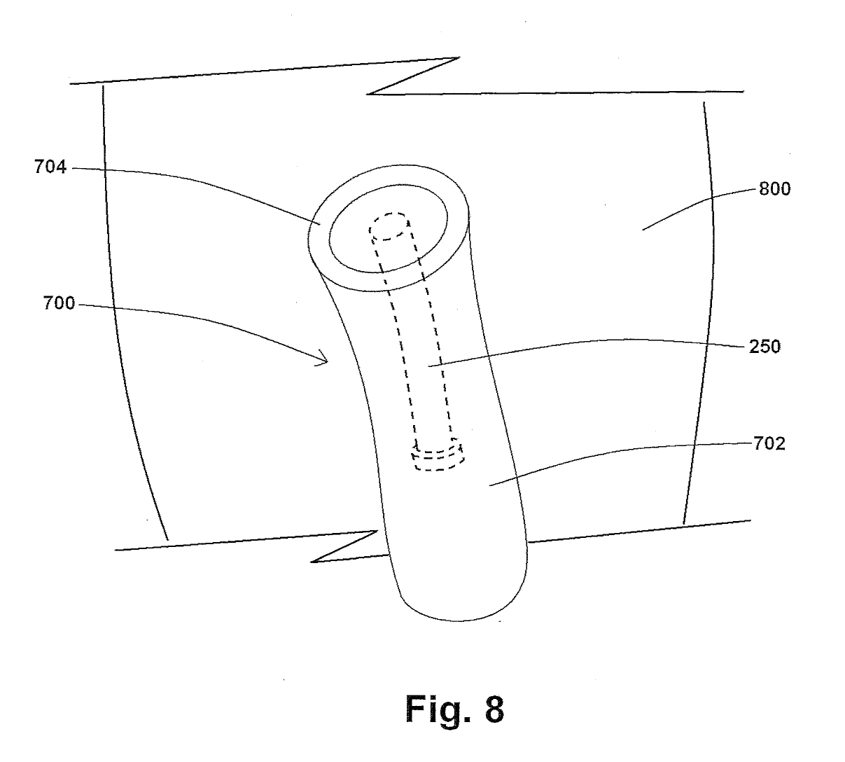

Such an adhesive device 700 may be useful, for example, when a patient wishes to take a shower but not introduce bacteria or other infectious agents to his or her bodily catheters. For example, and as shown in FIG. 8, a bodily catheter 250 may be positioned within device 700, and adhesive ring 704 may be pressed onto a patient's body 800 to form a fluid and/or air tight seal or a substantially fluid and/or air tight seal.

The various embodiments of devices 100 of the present disclosure may comprise any number of suitable materials known the art useful with medical devices, such as various types of medical grade plastics. Furthermore, various embodiments of devices 100 may have any number of apertures 110 and/or notches 202 positioned thereon of various sizes and configurations so that, for example, one or more dialysis catheters or PICC lines may be positioned therein. In addition, the various embodiments of devices 100 may be suitable for single use (disposable) or for multiple uses.

While various embodiments of devices and methods for maintaining an aseptic catheter environment have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device for maintaining an aseptic catheter environment, the device comprising:
a first housing and a second housing, the first housing and the second housing coupled to one another by way of at least one coupler, the first housing and the second housing configured to engage one another when in a closed configuration and to define an interior space when in the closed configuration; and
at least one aperture and/or notch defined within at least one of the first housing and the second housing, the at least one aperture and/or notch sized and shaped to allow at least part of a catheter to be positioned therethrough;
wherein when the device is closed about the at least part of a catheter and wherein when an antiseptic fluid is positioned within the device, the at least part of a catheter positioned within the device can come in contact with the antiseptic fluid, creating an effective aseptic environment at the at least part of the catheter positioned within the device.

2. The device of claim 1, wherein the at least one coupler comprises a hinge coupled to the first housing and the second housing so that the first housing and the second housing hingedly move about one another.

3. The device of claim 1, wherein the at least one coupler comprises at least one clip, the at least one clip operable to reversibly secure the first housing to the second housing.

4. The device of claim 1, wherein at least one of the first housing and the second housing comprises a housing gasket positioned around at least a portion of a perimeter of the first housing and/or the second housing.

5. The device of claim 4, wherein the first housing comprises the housing gasket, and wherein the second housing comprises a groove positioned around at least a portion of a perimeter of the second housing, the groove configured to receive said housing gasket.

6. The device of claim 4, wherein the housing gasket provides a fluid-tight seal when the first housing and the second housing engage each other in the closed configuration.

7. The device of claim 1, further comprising at least one aperture gasket positioned at or near the at least one aperture and/or notch, wherein the at least one aperture gasket provides a fluid-tight seal when a catheter is positioned within said aperture and/or notch.

8. The device of claim 1, further comprising one or more saturable materials positioned within the interior space of the device.

9. The device of claim 8, wherein the one or more saturable materials are selected from the group consisting of a sponge, a fabric, and/or a gauze.

10. The device of claim 1, further comprising:
a fluid sac containing an antiseptic fluid, the fluid sac positioned upon at least one of the first housing and the second housing, so that when the first housing and the second housing engage one another in the closed configuration, the fluid sac is positioned in the interior space.

11. The device of claim 10, wherein the fluid sac is capable of being ruptured by way of a rupturer positioned within said device.

12. The device of claim 11, wherein the rupturer is selected from the group consisting of a pin, a needle, a portion of the first housing, and a portion of the second housing.

13. The device of claim 11, wherein when the fluid sac becomes ruptured, the fluid sac releases fluid within the interior space of the device to create an aseptic environment within the interior space of the device.

14. The device of claim 10, wherein the fluid sac is capable of being ruptured by way of closing the device, whereby pressure from at least one of the first housing and the second housing causes the fluid sac to rupture.

15. The device of claim 14, wherein when the fluid sac becomes ruptured, the fluid sac releases fluid within the interior space of the device to create an aseptic environment within the interior space of the device.

16. The device of claim 1, wherein the at least one aperture and/or notch comprises at least two apertures and/or notches.

17. The device of claim 1, wherein the at least one aperture and/or notch comprises at least four apertures and/or notches, wherein at least two of the at least four apertures and/or notches are positioned relatively opposite to at least two other of the at least four apertures and/or notches, wherein when a portion of two catheters extending from a patient's body are coupled to two external catheters, the device is configured so that a portion of each said catheter extends from the device when the device is in the closed configuration.

18. The device of claim 1, further comprising:
a strap coupled thereto, the strap configured to be positioned about a portion of a patient's body.

19. (canceled)

20. A device for maintaining an aseptic catheter environment, the device comprising:
a first housing and a second housing, the first housing and the second housing coupled to one another by way of at least one hinge, the first housing and the second housing configured to engage one another when in a closed configuration and to define an interior space when in the closed configuration;
at least one clip coupled to the first housing or the second housing, the at least one clip operable to reversibly secure the first housing to the second housing;
one or more saturable materials positioned within the interior space of the device;
a fluid sac containing an antiseptic fluid, the fluid sac positioned upon at least one of the first housing and the second housing, so that when the first housing and the second housing engage one another in the closed configuration, the fluid sac is positioned in the interior space; and
at least one aperture and/or notch defined within at least one of the first housing and the second housing, the at least one aperture and/or notch sized and shaped to allow at least a portion of a catheter to be positioned therethrough.

21. A device for maintaining an aseptic catheter environment, the device comprising:
a first housing and a second housing, the first housing and the second housing coupled to one another by way of at least one hinge, the first housing and the second housing configured to engage one another when in a closed configuration and to define an interior space when in the closed configuration;
at least one clip coupled to the first housing or the second housing, the at least one clip operable to reversibly secure the first housing to the second housing;
one or more saturable materials positioned within the interior space of the device;
a fluid sac containing an antiseptic fluid, the fluid sac positioned upon at least one of the first housing and the second housing, so that when the first housing and the second housing engage one another in the closed configuration, the fluid sac is positioned in the interior space; and
at least four apertures and/or notches defined within at least one of the first housing and the second housing, wherein at least two of the at least four apertures and/or notches are positioned relatively opposite to at least two other of the at least four apertures and/or notches, the at least four apertures and/or notches sized and shaped to allow at least portions of catheters to be positioned therethrough.

22.-26. (canceled)
